(19) United States
(12) Patent Application Publication
(10) Pub. No.: US 2025/0197419 A1
(43) Pub. Date: Jun. 19, 2025

(54) PURIFICATION PROCESSES OF RIFAMPICIN FROM NITROSAMINES

(71) Applicant: OLON S.p.A., Rodano (MI) (IT)

(72) Inventors: Natwar PANCHAL, Mahad (MH) (IN); Madhu SADASHIVAIAH, Mahad (MH) (IN); Venu Babu GORIPARTHI, Mahad (MH) (IN); Sanjani VANGULE, Mahad (MH) (IN); Sandip PAWAR, Mahad (MH) (IN)

(21) Appl. No.: 18/862,437

(22) PCT Filed: May 12, 2023
(86) PCT No.: PCT/IB2023/054930

§ 371 Date: Nov. 1, 2024

(30) Foreign Application Priority Data
May 13, 2022 (IN) 202241027758

(51) Int. Cl. C07D 498/08

(52) U.S. Cl. CPC C07D 498/08

(57) ABSTRACT

A process for preparing rifampicin substantially free of 1-methyl-4-nitrosopiperazine (MeNP) may include: providing a mixture of crude rifampicin in an organic solvent, wherein the mixture is obtained by dissolving a solid crude rifampicin in the organic solvent; washing the mixture with an aqueous solution, having a pH greater than or equal to 2 and less than or equal to 7, so as to obtain an organic layer and an aqueous layer which are then separated; adding at least one antioxidant to the separated organic layer; distilling the organic solvent under inert atmosphere; crystallizing under the inert atmosphere; and isolating the rifampicin thus obtained under the inert atmosphere.

### TECHNICAL FIELD

The present invention relates to the control of the quantity of genotoxic impurities, in particular nitrosamines, in rifampicin.

In particular, the present invention relates to a process for the preparation of rifampicin substantially free of the 1-methyl-4-nitrosopiperazine (MeNP) impurity. The present invention also relates to a rifampicin substantially free of the 1-methyl-4-nitrosopiperazine (MeNP) impurity.

### PRIOR ART

Nitrosamines, which are organic compounds having general formula R₁R₂N—N═O, are classified by the EMA (European Medicines Agency) ICH M7 (R1) (Assessment and control of DNA reactive (mutagenic) impurities in pharmaceuticals to limit potential carcinogenic risk) as class 1 impurities, “known mutagenic carcinogens”, and by the International Agency for Research on Cancer (IARC) as 2A—“probable carcinogens”.

Such compounds are known by-products of various synthetic processes, for example in the presence of nitrosating agents (such as nitrite salts in acidic conditions) and amines (secondary, tertiary or quaternary), and can be difficult to separate: traces of these impurities may therefore be present in the finished product even after purification.

Traces of nitrosamines have been found in many active pharmaceutical ingredients for use in humans. In recent years, this has led global regulatory agencies, such as the FDA (Food and Drug Administration) and EMA, to impose stringent limits on the tolerance thresholds for these impurities. Consequently, the companies holding Marketing Authorization of drugs containing active pharmaceutical ingredients (API), of chemical or biological origin, need an increasingly accurate and stringent review of their medicinal products and production processes thereof, in order to prevent or limit the nitrosamine content. The attention paid by regulatory agencies to the issue of genotoxic impurities, with particular reference to nitrosamines, represents a precautionary approach aimed at guaranteeing the safety and health of the patient (in line with the measures introduced by the revision of article 5, paragraph 3, of EMA Regulation (EC) No 726/2004, to limit the presence of nitrosamines in medicinal products for human use).

Rifampicin (shown in the figure below) belongs to the rifamycin class and is an antibiotic used primarily in the treatment of tuberculosis, as well as in the treatment of severe infections, such as bloodstream infections and leprosy.

Rifampicin can for example be obtained starting from rifamycin-S, via an oxazino intermediate, typically not isolated, and subsequent reaction with 1-amino-4-methylpiperazine (1-AMP), to obtain rifampicin (see synthetic scheme below). Synthetic processes of this type are known in the literature, see for example U.S. Pat. No. 4,174,320 and CN101486716.

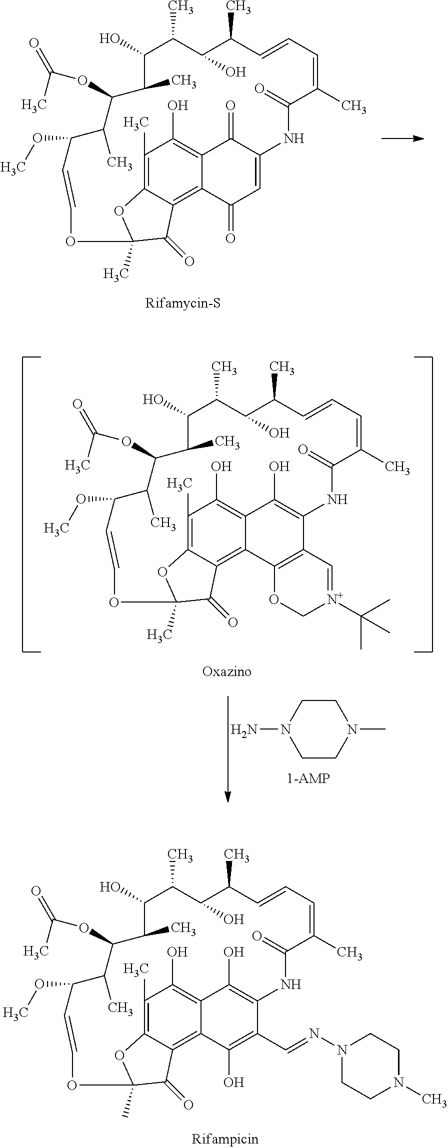

Traces of nitrosamines, particularly 1-methyl-4-nitrosopiperazine (MeNP), have recently been detected in commercially available batches of rifampicin. It is known that the presence of this genotoxic impurity derives from the use of 1-AMP in the production process of rifampicin, as well as from the oxidation of degradation products of rifampicin itself.

The FDA proposes 96 ng/day as the maximum daily intake of MeNP. With particular reference to rifampicin, from this data and considering the maximum daily dose which corresponds to 600 mg/day, the maximum MeNP threshold considered acceptable in the active ingredient corresponds to 0.16 parts per million (ppm; Sandrine Cloëz and Mike Frick, N-nitrosamines and Tuberculosis Medicines Rifampicin and Rifapentine, February 2021).

In fact, in August 2020 the FDA issued a note in which the acceptable limits of MeNP in rifampicin were set at 0.16 ppm, however specifying that the temporary distribution of rifampicin containing MeNP lower than 5 ppm is accepted in order to maintain the access for patients to these life-saving drugs.

To the best of the Applicant's knowledge, processes for obtaining rifampicin containing an amount of MeNP equal to or lower than 0.16 ppm are not known in the art.

Patent document CN111018887 describes a rifampicin purification process which allows to obtain a rifampicin with purity around 99.5% and with a single impurity content lower than 0.1%. This document, however, is completely silent with respect to the presence of genotoxic impurities, in particular MeNP, and therefore to the control of their amount.

### SUMMARY OF THE INVENTION

The Applicant has therefore tackled the aim of obtaining rifampicin substantially free of 1-methyl-4-nitrosopiperazine (MeNP), and in particular with a MeNP content equal to or lower than 0.16 ppm.

The Applicant has also tackled the aim of developing a purification process of rifampicin from genotoxic substances, in particular from 1-methyl-4-nitrosopiperazine (MeNP). Therefore, in a first aspect, the present invention relates to a process for the preparation of rifampicin substantially free of 1-methyl-4-nitrosopiperazine (MeNP), and preferably with a MeNP content equal to or lower than 0.16 ppm, said process comprising the steps of:
a) providing a mixture of crude rifampicin in an organic solvent, wherein said mixture is obtained by dissolving a solid crude rifampicin in an organic solvent;
b) washing said mixture with an aqueous solution having a pH from 2 to 7, so as to obtain an organic layer and an aqueous layer which are then separated;
c) adding at least one antioxidant to the separated organic layer obtained from step b);
d) distilling said organic solvent under inert atmosphere;
e) crystallizing under inert atmosphere; and
f) isolating rifampicin thus obtained under inert atmosphere.

The present invention also relates to a rifampicin with a 1-methyl-4-nitrosopiperazine (MeNP) content equal to or lower than 0.16 ppm.

### DETAILED DESCRIPTION OF THE INVENTION

### Definitions

In the present text, the terms “crude active ingredient” and “crude rifampicin” are intended to mean an active ingredient, in particular rifampicin, obtained from a preparation process prior to the process according to the present invention. In particular, it is intended to refer to rifampicin comprising one or more impurities, in particular comprising 1-methyl-4-nitrosopiperazine (MeNP).

For the purposes of the present invention, the term “substantially free of 1-methyl-4-nitrosopiperazine (MeNP)” is intended to refer to an active ingredient, in particular a rifampicin, comprising 1-methyl-4-nitrosopiperazine (MeNP) in quantities equal to or lower than 0.16 ppm.

For the purposes of the present invention, the term “process for the preparation of rifampicin substantially free of 1-methyl-4-nitrosopiperazine (MeNP)” is intended to refer to a purification process which has the purpose of removing, or reducing, the amount of impurities, in particular MeNP, from the crude active ingredient. Such process is applied downstream of the preparation process of the crude active ingredient.

Furthermore, such process can also be used to control the level of impurities that are formed by degradation of the crude active principle. In the present text, the term “to control/controlling” is intended to also include the terms “prevent” and “inhibit”.

The percentage values (%) indicated in the text, unless otherwise specified, are to be intended as percentages by weight.

For the purposes of the present invention, the term “aqueous solution having a pH from 2 to 7” is intended to refer to an aqueous solution at controlled pH which can comprise a pH adjusting agent or can be pure water when the pH is equal to 7.

### DETAILED DESCRIPTION

Therefore, in a first aspect, the present invention relates to a process for the preparation of rifampicin substantially free of 1-methyl-4-nitrosopiperazine (MeNP), said process comprising the following steps of:
a) providing a mixture of crude rifampicin in an organic solvent, wherein said mixture is obtained by dissolving a solid crude rifampicin in an organic solvent;
b) washing said mixture with an aqueous solution having a pH from 2 to 7, so as to obtain an organic layer and an aqueous layer which are then separated;
c) adding at least one antioxidant to the separated organic layer obtained from step b);
d) distilling said organic solvent under inert atmosphere;
e) crystallizing under inert atmosphere; and
f) isolating rifampicin thus obtained under inert atmosphere.

Preferably said process allows to obtain rifampicin with a MeNP content equal to or lower than 0.16 ppm.

According to a preferred aspect, said process allows to obtain rifampicin with a MeNP content from 0.01 to 0.16 ppm, preferably from 0.01 to 0.10 ppm, even more preferably from 0.01 to 0.05 ppm.

According to another preferred aspect, said process allows to obtain rifampicin with a MeNP content from 0.02 to 0.16 ppm, preferably from 0.02 to 0.10 ppm, even more preferably from 0.02 to 0.05 ppm.

According to a preferred aspect of the invention, said mixture provided in step a) is the crude mixture obtained at the end of a process of preparation of rifampicin.

According to an alternative aspect of the present invention, said mixture provided in step a) is a mixture obtained by dissolving a solid crude rifampicin in an organic solvent.

According to a preferred aspect of the invention the organic solvent used in step a) is selected from polar and non-polar aprotic solvents such as dichloromethane, ethyl acetate, 2-methyl tetrahydrofuran, methyl isobutyl ketone, toluene and mixtures thereof. According to a preferred aspect, said organic solvent is a non-polar aprotic solvent. Preferably said non-polar aprotic solvent is dichloromethane.

The step b) of the process according to the present invention is a liquid-liquid extraction phase in which said aqueous solution is added to the mixture of step a) obtaining a biphasic mixture; the two organic and aqueous layers are mixed, for example by stirring, and subsequently separated. The aqueous layer is then discarded and the organic layer is used in step c) of the above process. Preferably, step b) can be repeated several times, for example 2-5 times: in this case at the end of each step b) the organic layer obtained after the first washing is recovered and washed again with said aqueous solution.

Step b) is carried out in order to remove any excess of unreacted 1-AMP and MeNP, both being water-soluble compounds, therefore at the end of this step a mixture comprising said organic solvent and rifampicin with a reduced, but not null, content of impurities, is obtained.

According to another preferred aspect of the invention, the process also comprises one or more additional washing phases, subsequent to step b). Preferably, said one or more additional washing steps are carried out with an aqueous solution, more preferably with water.

According to a preferred aspect of the invention the aqueous solution used has neutral pH, preferably it is pure water at pH 7.

According to another preferred aspect of the invention, the aqueous solution used in step b) is water having a controlled pH, also defined as acidic water, having a pH from 2 to 6, preferably from 3 to 6, even more preferably from 3 to 5.

Preferably, this aqueous solution comprises a pH adjusting agent selected from organic and inorganic acids. Preferably said pH adjusting agent is an organic acid or a mixture of organic acids, more preferably selected from acetic acid, formic acid, oxalic acid, and mixtures thereof. Preferably, the pH adjusting agent is acetic acid.

Advantageously, without binding to any theory, the addition of at least one antioxidant in step c), before distillation (step d), allows the inhibition of the formation of oxygen free radicals, thus avoiding the new formation of MeNP.

Preferably said at least one antioxidant is selected from ascorbic acid, ascorbyl-2-glucoside, ascorbyl-6-octanoate, ascorbyl-6-palmitate, cysteine, sodium metabisulphite, propyl gallate, butylhydroxyanisole, butylhydroxytoluene (BHT) and combinations thereof. In a particularly preferred aspect of the present invention said at least one antioxidant is ascorbic acid.

According to another particularly preferred aspect of the present invention said at least one antioxidant is a mixture of ascorbic acid and butylhydroxytoluene (BHT).

Preferably, said antioxidant is added in an amount of from 0.3% to 2% by weight, preferably from 0.5% to 1%. The amount of the antioxidant is calculated as a percentage by weight with respect to the weight of rifamycin S, the starting material used for the synthesis of rifampicin. Alternatively, in the embodiments where the mixture a) is provided starting from solid crude rifampicin, said percentage is calculated by weight with respect to the weight of the crude rifampicin used.

The steps d)-f), and preferably also possible further optional steps preceding or following any of the steps d)-f), are carried out in an inert atmosphere in order to prevent, or limit, the new formation of MeNP. Preferably, the inert atmosphere is an atmosphere substantially free of oxygen, for example substantially saturated with an inert gas such as nitrogen or argon, or under vacuum.

According to a preferred aspect said inert atmosphere is an atmosphere with an amount of 02 lower than 3% by volume, preferably lower than 1% by volume, even more preferably equal to or lower than 0.5% by volume.

According to another preferred aspect, said inert atmosphere has a relative oxygen density <0.1.

Steps d)-f) are carried out according to methods known in the art.

In particular, the distillation step d) involves the use of temperatures dependent on the boiling temperature of the organic solvent selected for step a). Typically, at the end of step d) a concentrated residue is obtained, from which essentially all the organic solvent has been eliminated.

According to a preferred aspect of the present invention, the process involves a further step d′), preferably also to be carried out in inert atmosphere, following step d), in which an aprotic polar solvent, preferably acetone, is added in the residue obtained from the distillation step d) and the resulting solution is distilled again. This step is, for example, useful for eliminating possible traces of the organic solvent used in step a) before carrying out the crystallization step e).

Preferably the crystallization step e) is carried out using an aprotic polar solvent, preferably acetone or an acetone/ethyl acetate mixture. Preferably in step e) the residue obtained from step d) or d′) is dissolved in the selected aprotic polar solvent, preferably pre-heated, the mixture obtained is heated, typically at the reflux temperature of the selected solvent, and subsequently slowly cooled.

Advantageously the isolation step f) is carried out by filtration, for example filtration on Buchner filter optionally under vacuum. At the end of this step, solid rifampicin is obtained.

According to a preferred aspect of the present invention, the process involves a further step f), following step f), which involves washing the solid rifampicin obtained after crystallization with aprotic polar solvent, preferably acetone, and again isolating the rifampicin thus obtained. Preferably the step f) is carried out in inert atmosphere.

According to another preferred aspect of the present invention, the process involves a further step g), following the isolation step f) or f′), in which the obtained rifampicin is dried, preferably in inert atmosphere.

In a second aspect, the present invention therefore relates to a rifampicin characterized in that it is substantially free of 1-methyl-4-nitrosopiperazine (MeNP), and preferably comprises an amount of MeNP lower than or equal to 0.16 ppm.

Another aspect of the present invention concerns a rifampicin characterized by the fact of being substantially free of 1-methyl-4-nitrosopiperazine (MeNP), and preferably of comprising a quantity of MeNP lower than or equal to 0.16 ppm, obtainable by the above process described.

Preferably said rifampicin comprises an amount of 1-methyl-4-nitrosopiperazine (MeNP) from 0.01 to 0.16 ppm, preferably from 0.01 to 0.10 ppm, even more preferably from 0.01 to 0.05 ppm.

According to another preferred aspect, said rifampicin comprises an amount of MeNP from 0.02 to 0.16 ppm, preferably from 0.02 to 0.10 ppm, even more preferably from 0.02 to 0.05 ppm.

The above is intended as an example and not as a limitation. Furthermore, the skilled person will be able to understand that modifications can be made without departing from the scope of the present invention.

### EXPERIMENTAL PART

The present description will be better illustrated in the following examples which have only an illustrative and not limitative purpose.

### Example 1—Preparation of Crude Rifampicin Starting from Rifamycin-S

Rifamycin S (105 g, 0.1509 mol) was dissolved in N,N-dimethylformamide (53.0 g) and dichloromethane (40 ml), heated to a temperature of about 35-40° C. and stirred until completely dissolved. Acetic acid (25 g, 0.4163 mol) and N-methyl-tert-butylamine (32 g, 0.3671 mol) were then added, and the mixture was kept for 6 hours at a temperature of about 32-36° C.

The mixture is then cooled to room temperature, dichloromethane (108 ml), piperazine (20 g, 0.2322 mol) and 1-amino-4-methyl piperazine (32 g, 0.2778 mol) are added, and the mixture was kept at a temperature of about 32-36° C. for about 3 hours, until completion of the reaction (as evidenced by TLC).

The mixture was then washed with an aqueous solution at pH 5-6. 1-amino-4-methyl piperazine (0.32 g) was added to the organic layer and the mixture brought to reflux temperature and stirred for 2 hours.

A mixture of crude rifampicin in dichloromethane is thus obtained.

### Example 2—Purification of Crude Rifampicin (According to the Invention)

The mixture of crude rifampicin in dichloromethane obtained according to example 1 was subjected to washing with an aqueous solution of acetic acid at pH 3.9-4.2 (540 ml) and subsequent stirring for 30 minutes. The mixture was then left to rest for 60 minutes and subsequently the organic phase was separated from the aqueous one.

The mixture thus obtained was heated to a temperature of about 45° C. and butylhydroxytoluene (0.1 g, 4.5*10\hat{ }⁻⁴ moles) and ascorbic acid (0.52 g, 0.003 moles, 0.5% by weight with respect to the weight of the rifamycin S used in Example 1) were added. The dichloromethane was then distilled at about 53° C. in inert atmosphere (under nitrogen saturation conditions).

The residue thus obtained was then dissolved, again in inert atmosphere, in previously heated acetone (200 ml) and the mixture was subjected to a second distillation in inert atmosphere, at a temperature of about 50-58° C.

The residue thus obtained was then crystallized with acetone in inert atmosphere (under nitrogen saturation conditions). In particular, under inert atmosphere, the residue was dissolved in acetone (520 ml) preheated to 53° C., the mixture was heated to reflux (about 55-59° C.) and kept at this temperature for 40-45 minutes, then slowly cooled down to a temperature between −5 and −20° C.

Pure rifampicin was then isolated by filtration in inert atmosphere (under nitrogen saturation conditions) and washed with 80 ml of cold acetone. The solid rifampicin obtained was then vacuum dried at 55° C.

### Example 3—Purification of Crude Rifampicin (According to the Invention)

The procedure of Example 2 was repeated using 1% of ascorbic acid (1.04 g, 0.006 moles, by weight with respect to the weight of rifamycin S used in Example 1) instead of 0.5%.

### Example 4—Purification of Crude Rifampicin (Comparison)

The procedure of Example 2 was repeated without using ascorbic acid or inert atmosphere in any of the steps described.

### MeNP Content in Purified Rifampicin

Table 1 below shows the MeNP content, determined by liquid chromatography-mass spectrometry (LC-MS), detected in the products obtained from examples 2-4.

TABLE 1

| | MeNP content |
|---|---|
| Sample | determined by LC-MS |
| 2 | 0.033 ppm |
| 3 | 0.028 ppm |
| 4 | 0.250 ppm |

From the data summarized in Table 1 it is evident that the use of ascorbic acid and inert atmosphere in the purification of rifampicin are necessary to obtain rifampicin comprising very low quantities of MeNP (samples 2-3), in particular quantities well below the limit set by regulatory agencies of 0.16 ppm.

In fact, by carrying out the same purification process without the use of ascorbic acid or inert atmosphere (comparison sample 4), a rifampicin is obtained comprising a MeNP content 1.5 times higher than the limits established by the FDA, and almost 10 times higher to that obtained through the process according to the present invention.

1. A process for preparing rifampicin substantially free of 1-methyl-4-nitrosopiperazine (MeNP), the process comprising:
providing a mixture of crude rifampicin in an organic solvent, wherein the mixture is obtained by dissolving a solid crude rifampicin in the organic solvent;
washing the mixture with an aqueous solution, having a pH greater than or equal to 2 and less than or equal to 7, so as to obtain an organic layer and an aqueous layer which are then separated;
adding at least one antioxidant to the separated organic layer;
distilling the organic solvent under inert atmosphere;
crystallizing under the inert atmosphere; and
isolating the rifampicin thus obtained under the inert atmosphere.

2. The process of claim 1, wherein the rifampicin thus obtained comprises the 1-methyl-4-nitrosopiperazine in an amount less than or equal to 0.16 parts per million (ppm).

3. The process of claim 1, wherein the rifampicin thus obtained comprises the 1-methyl-4-nitrosopiperazine in an amount greater than or equal to 0.01 parts per million (ppm) and less than or equal to 0.16 ppm.

4. The process of claim 1, wherein the organic solvent provided in the mixture is selected from polar and non-polar aprotic solvents.

5. The process of claim 1, wherein the organic solvent provided in the mixture comprises dichloromethane.

6. The process of claim 1, wherein the at least one antioxidant is selected from ascorbic acid, ascorbyl-2-glucoside, ascorbyl-6-ottanoate, ascorbil-6-palmitate, cysteine, sodium metabisulfite, propyl gallate, buthyilidroxyanisole, butylhydroxytoluene (BHT), or combinations thereof.

7. The process of claim 1, wherein the at least one antioxidant comprises ascorbic acid.

8. The process of claim 1, wherein the inert atmosphere is a substantially nitrogen-saturated atmosphere, is a substantially argon-saturated atmosphere, or is under vacuum.

9. The process of claim 1, wherein isolating the rifampicin is carried out by filtration.

10. The process of claim 1, further comprising:
washing the rifampicin thus obtained with aprotic polar solvent; and
isolating the washed rifampicin.

11. The process of claim 1, further comprising:
drying the rifampicin thus obtained.

12. A rifampicin prepared by the process of claim 1, wherein an amount of 1-methyl-4-nitrosopiperazine in the rifampicin is less than or equal to 0.16 parts per million (ppm).

13. A rifampicin that comprises an amount of 1-methyl-4-nitrosopiperazine less than or equal to 0.16 parts per million (ppm).

14. The process of claim 1, wherein the rifampicin thus obtained comprises 1-methyl-4-nitrosopiperazine in an amount greater than or equal to 0.01 parts per million (ppm) and less than or equal to 0.10 ppm.

15. The process of claim 1, wherein the organic solvent provided in the mixture comprises one or more polar aprotic solvents.

16. The process of claim 1, wherein the organic solvent provided in the mixture comprises one or more non-polar aprotic solvents.

17. The process of claim 1, wherein the organic solvent provided in the mixture comprises dichloromethane, ethyl acetate, 2-methyl tetrahydrofuran, methyl isobutyl ketone, toluene, or mixtures thereof.

18. The process of claim 1, wherein the at least one antioxidant comprises one or more of ascorbic acid, ascorbyl-2-glucoside, ascorbyl-6-ottanoate, ascorbil-6-palmitate, cysteine, sodium metabisulfite, propyl gallate, buthyilidroxyanisole, and butylhydroxytoluene (BHT).

19. The process of claim 1, wherein an amount of O₂ in the inert atmosphere is less than 3% by volume.

20. The process of claim 10, further comprising:
drying the washed and isolated rifampicin.
